(19) United States
(12) Patent Application Publication
(10) Pub. No.: US 2021/0005280 A1
(43) Pub. Date: Jan. 7, 2021

(54) VARIANT CALLING USING MACHINE LEARNING

(71) Applicant: MYRIAD WOMEN'S HEALTH, INC., South San Francisco, CA (US)

(72) Inventors: Kyle BEAUCHAMP, San Francisco, CA (US); Dale MUZZEY, San Francisco, CA (US); Adithya C. GANESH, Stanford, CA (US); Sun Hae HONG, San Carlos, CA (US)

(73) Assignee: MYRIAD WOMEN'S HEALTH, INC., South San Francisco, CA (US)

(21) Appl. No.: 17/028,303

(22) Filed: Sep. 22, 2020

Related U.S. Application Data
(63) Continuation of application No. PCT/US2019/022712, filed on Mar. 18, 2019.
(60) Provisional application No. 62/646,784, filed on Mar. 22, 2018.
(60) Provisional application No. 62/664,620, filed on Apr. 30, 2018.

(51) Int. Cl. G16B 20/20; G06K 9/62; G06N 3/04; C12Q 1/6869; G16B 40/00

(52) U.S. Cl. CPC G16B 20/20; G06K 9/6256; C12Q 2600/156; C12Q 1/6869; G16B 40/00; G06N 3/04

(57) ABSTRACT

Methods for determining a respective carrier status of an individual are disclosed. In some examples, the method includes determining the respective carrier status based on copy number data for a gene in a genome of the individual and SNP data for the gene using a machine learning algorithm. In some examples, the machine learning algorithm is configured to receive, as inputs, the copy number data and the SNP data, and output the respective carrier status of the individual.

### CROSS-REFERENCE TO RELATED APPLICATIONS

This application is a continuation of International Application No. PCT/US2019/022712, filed Mar. 18, 2019, which claims the benefit of priority to U.S. Provisional Application No. 62/646,784, filed Mar. 22, 2018, and to U.S. Provisional Application No. 62/664,620, filed Apr. 30, 2018, each of which is incorporated herein by reference in their entirety.

### FIELD OF THE DISCLOSURE

This relates generally to identifying a genetic condition from sequenced genomes (or portions of genomes).

### BACKGROUND OF THE DISCLOSURE

Certain genetic conditions can be associated with the number of functional copies of one or more genes and/or single nucleotide polymorphisms in an individual's genome. As such, identification of such genetic conditions can be accomplished using information about the above, and a method of determining such genetic conditions, while reducing the need for human involvement in making such determinations, is desirable.

The disclosures of all publications, patents, and patent applications referred to herein are each hereby incorporated by reference in their entireties. To the extent that any reference incorporated by reference conflicts with the instant disclosure, the instant disclosure shall control.

### SUMMARY OF THE DISCLOSURE

Various genetic conditions can be associated with an individual having fewer than two functional copies of a specific gene in their genome (e.g., for autosomal dominant conditions, such as Lynch syndrome), or an individual having fewer than one functional copy of a specific gene in their genome (e.g., for autosomal recessive conditions). For example, an individual's lack of a functional copy of the CYP21A2 gene can lead to the individual having congenital adrenal hyperplasia (CAH). Data relating to the number of copies of genetic material corresponding to the gene of interest in the individual's genome, and data relating to the number of sequencing reads from a location in the gene of interest in the individual's genome that have a single nucleotide polymorphism at that location can be used to determine whether the individual has two (or one, or none) functional copies of the gene of interest and/or the nature of mutations in the gene of interest, if any. The examples of the disclosure provide various ways in which a machine learning algorithm can be used to make such determinations.

### BRIEF DESCRIPTION OF THE DRAWINGS

FIG. 1 illustrates the existence of an exemplary gene and pseudogene in a genome (or a portion of the genome) of a healthy human according to examples of the disclosure.

FIG. 2A illustrates a scenario in which an individual does not have two functional copies of the gene of interest (e.g., CYP21A2 gene) according to examples of the disclosure.

FIG. 2B illustrates example copy number data and SNP data that might be obtained by sequencing a gene and pseudogene of the individual corresponding to the sample discussed in FIG. 2A.

FIG. 3A illustrates an exemplary process in which an RNN can be used to determine one or more carrier statuses associated with the genome (or portion of the genome) being sequenced according to examples of the disclosure.

FIG. 3B illustrates an alternative flow for the process of FIG. 3A in which an RNN only outputs variant calls if those calls are associated with relatively high confidence levels according to examples of the disclosure.

FIG. 3C illustrates an exemplary process in which anomaly detection is used before data is inputted to an RNN for determining one or more carrier statuses associated with the genome being sequenced according to examples of the disclosure.

FIG. 3D illustrates another exemplary process for determining one or more carrier statuses associated with the genome (or portion of the genome) being sequenced in which anomaly detection and flagging for review are utilized according to examples of the disclosure.

FIGS. 4A-4B illustrate exemplary details of a RNN that can be utilized for determining one or more carrier statuses associated with the genome (or portion of the genome) being sequenced according to examples of the disclosure.

FIG. 5 illustrates exemplary structures of SNP, copy number and carrier status data according to examples of the disclosure.

FIG. 6 illustrates an exemplary computing system or electronic device for implementing the examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

Various genetic conditions can be associated with an individual having fewer than two functional copies of a specific gene in their genome (e.g., for autosomal dominant conditions, such as Lynch syndrome), or an individual having fewer than one functional copy of a specific gene in their genome (e.g., for autosomal recessive conditions). For example, an individual's lack of a functional copy of the CYP21A2 gene can lead to the individual having congenital adrenal hyperplasia (CAH). Data relating to the number of copies of genetic material corresponding to the gene of interest in the individual's genome, and data relating to the number of sequencing reads from a location in the gene of interest in the individual's genome that have a single nucleotide polymorphism at that location can be used to determine whether the individual has two (or one, or none) functional copies of the gene of interest and/or the nature of mutations in the gene of interest, if any. The examples of the disclosure provide various ways in which a machine learning algorithm can be used to make such determinations.

FIG. 1 illustrates the existence of an exemplary gene and pseudogene in a genome (or a portion of the genome) of a healthy human according to examples of the disclosure. Specifically, as mentioned above, a healthy human generally has two functional copies of a given gene—one copy on a maternal chromosome and one copy on a paternal chromosome. The individual also generally includes a maternal copy and a paternal copy of a pseudogene corresponding to the gene of interest, where the gene and the pseudogene can have coding regions that are on the order of 95% identical or more (e.g., 96%, 97%, 98%, or 99% or more) within the exon coding region. Thus, as shown in FIG. 1, a healthy human can have chromosome 102A and chromosome 102B, where chromosome 102A includes the gene of interest 104A and a corresponding pseudogene 106A, and chromosome 102B also includes the gene of interest 104B and a corresponding pseudogene 106B. Although FIG. 1 indicates the gene of interest and the corresponding pseudogene on the same chromosome, it is understood that, for certain gene/pseudogene pairs, the gene and the corresponding pseudogene are on different chromosomes (e.g., SDHA). In the example of FIG. 1, the gene of interest is a CYP21A2 gene, which has a corresponding CYP21A1P pseudogene. While some of the examples of the disclosure are described with reference to the CYP21A2 gene and the CYP21A1P pseudogene, it is understood that the techniques of the disclosure can also apply to other gene-pseudogene pairs. Some exemplary gene-pseudogene pairs, and associated genetic conditions, include: 1) CYP21A2 (gene) and CYP21A1P (pseudogene) associated with CAH; 2) GBA (gene) and psGBA (pseudogene) associated with Gaucher disease; 3) PMS2 (gene) and PMS2CL (pseudogene) associated with Lynch Syndrome; and 4) SMN1 (gene) and SMN2 (pseudogene) associated with spinal muscular atrophy.

If an individual does not have the requisite number (e.g., two or one) of functional copies of the gene of interest (e.g., genes 104A and 104B), that individual may exhibit any of several inherited genetic conditions. For example, in reference to the CYP21A2 gene, the individual's lack of at least one functional copies of that gene can lead to the individual having congenital adrenal hyperplasia (CAH). Furthermore, the presence of only a single functional copy of the CYP21A2 gene indicates that this person is a carrier. If two carriers of an autosomal recessive condition have a child, the child has a 25% chance of inheriting zero functional copies and thus being affected. Thus, it can be beneficial to accurately determine whether an individual does not have two functional copies of the gene of interest so as to be able to diagnose that individual as carrying a corresponding genetic condition. Specifically, the examples of the disclosure can be used to identify any one or more of the following: two functional copies of the gene of interest; one functional copy of the gene of interest, one non-functional copy of the gene of interest (e.g., due to a mutation at one or more locations in the gene); less than two copies of the gene of interest (e.g., only one copy of the gene of interest) and/or whether those copies are functional or non-functional; more than two copies of the gene of interest (e.g., three copies of the gene of interest) and/or whether those copies are functional or non-functional, etc. Further, it is understood that while some of the examples of the disclosure are provided in the context of determining whether an individual has CAH by determining one or more characteristics of the individual's CYP21A2 genes, the examples of the disclosure can be used to diagnose other genetic conditions related to other genes (and/or pseudogenes) in analogous manners, as mentioned above.

In some examples, whether an individual has two functional copies of the gene of interest (e.g., the CYP21A2 gene) can be determined using “copy number data” and “single nucleotide polymorphism (SNP) data” relating to the gene of interest and/or the corresponding pseudogene (e.g., the CYP21A1P pseudogene) in the individual's genome. In some examples of the disclosure, “SNP data” can be data associated with a given location in the gene and/or the pseudogene of interest that is indicative of the number of sequencing reads from a sample that have a deleterious SNP (relative to a reference genome, a reference portion of a genome or a reference sequence) at that location. For example, the SNP data can be a ratio of the number of sequencing reads that detected a SNP at that location to the number of sequencing reads that did not detect a SNP at that location, or a ratio of the number of sequencing reads that detected a SNP at that location to the total number of sequencing reads obtained at that location (whether or not those reads detected a SNP at that location). In some examples, the SNP data can be count data and/or fraction data indicative of the relative abundance of the wild type versus mutant base at each locus, and in some examples, and in some examples can also include SNP call data that can be binary (e.g., indicating that a particular location is wild type or mutant) or descriptive (e.g., indicating that a particular location has a particular nucleotide). In some examples of the disclosure, “copy number data” can be data that indicates the number of copies of genetic material corresponding to the gene of interest and/or the corresponding pseudogene that are detected, on average, during sequencing of the individual's genome at various locations (e.g., single base pair locations or regions, such as clusters of base pairs) in the genome.

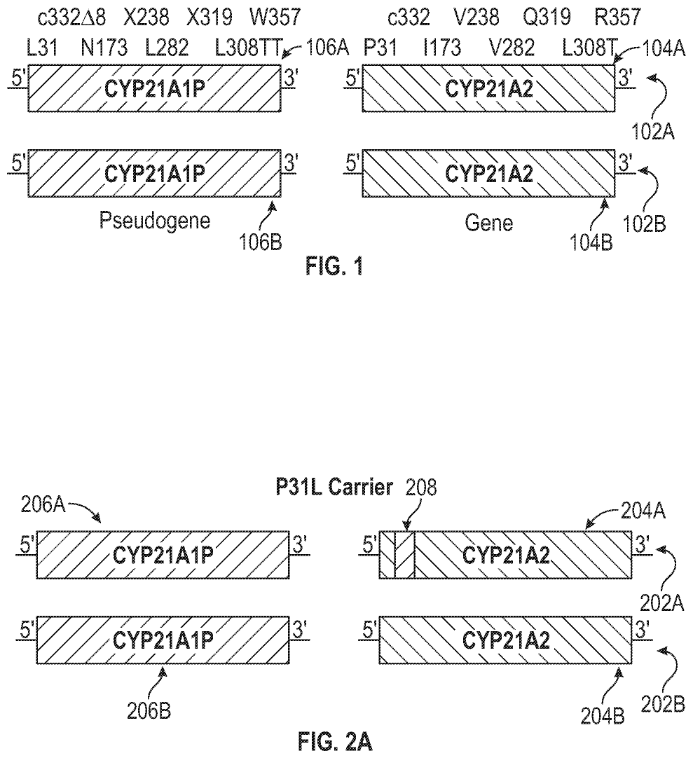

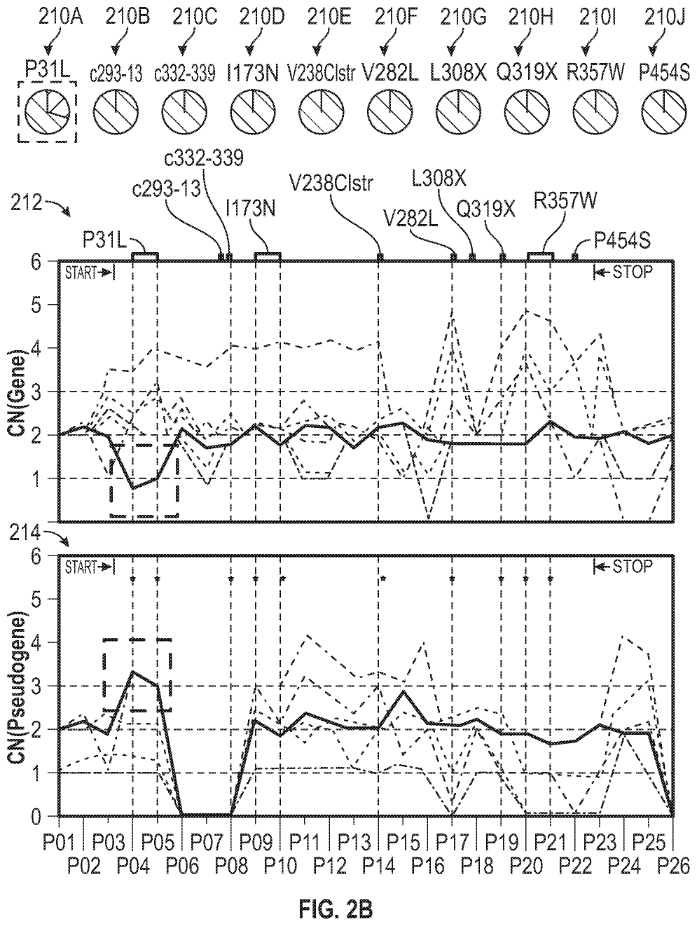

FIG. 2A illustrates a scenario in which an individual does not have two functional copies of the gene of interest (e.g., CYP21A2 gene) according to examples of the disclosure. Specifically, this individual has a normal copy of the CYP21A2 gene 204B and a copy of the CYP21A1P pseudogene 206B in chromosome 202B; however, the CYP21A2 gene 204A in chromosome 202A has a mutation at location 208 in the CYP21A2 gene 204A that results in the genomic sample being a P31L carrier, and thus a potential cause for CAH. Identification of this fact (e.g., one functional copy of the gene of interest and one non-functional copy of the gene of interest) can be accomplished pursuant to the examples of the disclosure, as will be described below.

FIG. 2B illustrates example copy number data and SNP data that might be obtained by sequencing a gene and pseudogene of the individual corresponding to the sample discussed in FIG. 2A. It is understood that the copy number data and the SNP data can be obtained using any suitable genetic sequencing methodology, such as whole genome sequencing (e.g., for SNP and/or copy number data), targeted sequencing with biotin capture (e.g., for SNP and/or copy number data), MLPA (e.g., for copy number data) and targeted genotyping (e.g., for SNP data). In some examples, the copy number data and the SNP data can be obtained using direct targeted sequencing (DTS). Direct targeted sequencing uses a capture probe library comprising a plurality of capture probes that hybridize to nucleic acid molecules in the sequencing library. The capture probes are designed to hybridize to segments within the region of interest (e.g., the gene and/or pseudogene of interest), and each capture probe has a corresponding segment. The region of interest is therefore determined by the capture probes used to enrich the sequencing library. The capture probes are extended using the nucleic acid molecules hybridized to the capture probe as a template. The extended capture probe can then be sequenced to obtain the sequence of a portion (that is, the portion corresponding to the segment from the region of interest) of the nucleic acid molecule. Because the sequence of the capture probe itself is determined, the segment corresponding to the capture probe begins following the terminus of the capture probe. In some embodiments, the extended capture probe is amplified to obtain additional copies. Amplification of the extended capture probe can also introduce artifacts in the sequencing depth, which can be normalized. U.S. Pat. No. 9,309,556, entitled “Direct Capture, Amplification and Sequencing of Target DNA using Immobilized Primers”; U.S. Pat. No. 9,092,401, entitled “System and Method for Detecting Genetic Variation”; U.S. Patent App. No. 2014/0024541, entitled “Methods and Compositions for High-throughput Screening”; Myllykangas et al. “Efficient targeted resequencing of human germline and cancer genomes by oligonucleotide-selective sequencing.” Nat Biotechnol. 29(11):1024-7 (2011); and Hopmans et al., “A programmable method for massively parallel targeted sequencing.” Nucleic Acids Res. 42(10):e88 (2014) describe embodiments of direct targeted sequencing. Direct targeted sequencing need not be performed using surface-based methods, but can also be performed in solution.

Referring again to FIG. 2B, the sequencing data can include SNP data 210 corresponding to the gene of interest (e.g., CYP21A2) (and/or in some examples, the corresponding pseudogene (e.g., CYP21A1P)), copy number data 212 corresponding to the gene of interest and copy number data 214 corresponding to the corresponding pseudogene 214. In the context of the CYP21A2 gene and the CYP21A1P pseudogene (understanding that the below description would apply analogously to other gene/pseudogene pairs of interest), SNP data 210 can include SNP data for various predetermined locations within the CYP21A2 gene and/or the CYP21A1P pseudogene. In some examples, these predetermined locations can be locations in the CYP21A2 gene and/or the CYP21A1P pseudogene that are known to be associated with particular genetic conditions (e.g., P31L carrier, I173N carrier, Q319X carrier, etc.). For example, SNP data 210A can be SNP data corresponding to location 208 in the CYP21A2 gene (e.g., as shown in FIG. 2A) and/or the CYP21A1P pseudogene where the existence of a SNP can result in the genomic sample being a P31L carrier. The SNP data 210 can include additional SNP data (e.g., data 210B, 210C, etc.) from other locations (e.g., different and/or unique locations) within the gene and/or the pseudogene of interest that are associated with particular genetic conditions. As previously mentioned, in some examples, the SNP data associated with a given location in the gene and/or the pseudogene of interest can be indicative of the number of samples sequenced that have a SNP at that location. For example, the SNP data can be a ratio of the number of sequencing reads that detected a SNP at that location to the number of sequencing reads that did not detect a SNP at that location, or a ratio of the number of sequencing reads that detected a SNP at that location to the total number of sequencing reads obtained at that location (whether or not those reads detected a SNP at that location).

Copy number data 212 and 214 can indicate the number of copies of one or more segments of the CYP21A2 gene and/or the CYP21A1P pseudogene. The line plots in copy number data 212 and 214 can correspond to copy number data for the individual of interest. In some examples, copy number data for other patients can be used to assess the significance of the copy number data variation of one patient (e.g., the individual of interest) as compared to the noise level of typical samples on that flow cell (e.g., used as data against which the copy number data for the current sample is validated). The segments of the CYP21A2 gene and/or the CYP21A1P pseudogene to which copy number data 212 and 214 correspond can correspond to a specific genetic locus (e.g., a single base) or can correspond to a sequencing read arising from a probe targeted to a region of the gene or pseudogene. For example, in some examples, one or more sequencing probes can be used to sequence the genome at different positions within the CYP21A2 gene and/or the CYP21A1P pseudogene to obtain copy number data corresponding to given positions in those genes/pseudogenes. Because a given sequencing run can include noise from various sources (e.g., probes, DNA, etc.), the sequencing can be normalized based on GC content, read mappability to a reference genome, performance of other samples in a multiplexed sequencing run, or any other normalization method known in the art. In some examples, copy number data and/or SNP data for a given location can be determined from a reading of a single probe corresponding to that location, or from readings of multiple probes at different locations to create normalized copy number and/or SNP data for that given location. In some examples, copy number data 212 and 214 can be determined based on counts of probe reads of pair-end sequencing that are normalized within the sample, and across the sample, to give the copy number at each probe binding site. For example, in the example of FIG. 2B, the number of copies of CYP21A2 genetic material detected at the position corresponding to probe P01 can be 2, the number of copies of CYP21A2 genetic material detected at the position corresponding to probe P02 can be slightly more than 2 (e.g., 2.3), the number of copies of CYP21A2 genetic material detected at the position corresponding to probe P03 can be slightly more than 2 (e.g., 2.1), etc. However, in the case that the genome being sequenced corresponds to a P31L carrier, it can be the case that the number of copies of CYP21A2 genetic material detected at the positions corresponding to probes P04 and P05 can be substantially less than 2 (e.g., closer to 1, because the individual can be missing one copy of that gene segment at those positions within the gene or pseudogene). Correspondingly, it can be the case that the average number of copies of CYP21A1P genetic material detected at the positions corresponding to probes P04 and P05 can be substantially more than 2 (e.g., closer to 3, even above 3 as in FIG. 2B, because the individual can have an extra copy of that genetic material at those positions in their genome). In some examples, copy number data for the gene can be differentiated from copy number data for the pseudogene based on one or more base pairs of one or more probes (e.g., the 40-th base pair of the 39-mer probes). For example, if a probe terminates at position N−1 and the extended probe is sequenced to include position N, the base sequenced at position N can determine whether the copy number count for a given probe arises from the gene or pseudogene based on the expected base at position N. In some examples, the positions of the genome sequenced by the sequencing probes can be the same as the positions to which the SNP data 210 correspond, can be different than the positions to which the SNP data 210 correspond, and/or can be overlapping with the positions to which the SNP data 210 correspond (e.g., can include some of the same positions, and some different positions). In some examples, copy number data for one or more probes (e.g., locations) may only be available for one of the gene and the pseudogene—thus, copy number data for the other of the gene and the pseudogene at those probes (e.g., locations) may be zero or non-existent (e.g., as at probes P06, P07 and P08 in FIG. 2B).

The above-described SNP and copy number data can be obtained from a genomic sample of interest with the goal of determining the carrier status of the individual from which the genomic sample was collected, as described above. Different carrier statuses can be associated with different copy number and/or SNP data. For example, in the context of the CYP21A2 gene and the CYP21A1P pseudogene, P31L carrier status can be associated with the SNP and copy number data described with reference to FIGS. 2A-2B. Other carrier statuses (e.g., indications of the existence of one or more given genetic conditions) can be associated with different SNP and copy number data for the CYP21A2-CYP21A1P gene-pseudogene pair, or for other gene-pseudogene pairs of interest.

According to examples of the disclosure, machine learning algorithms can be used to receive as inputs SNP and/or copy number data, as described above, and output determinations relating to whether or not the sequenced genome is associated with one or more genetic conditions (e.g., output information about one or more carrier statuses of the individual). Some of the machine learning algorithms that can be used in accordance with the examples of the disclosure can be convolutional neural networks (CNNs) (e.g., which can be effective, because genetic data can be spatially correlated), support vector machines (SVMs), random forest, etc. Because DNA, and thus genes and pseudogenes of interest, can have a sequential character, recurrent neural networks (RNNs) can be especially conducive for use in such applications, because RNNs make use of sequential information in their operation in that the output of a RNN for a given element in a sequence depends on the operations of the RNN during the previous one or more elements in the sequence—such operation that is grounded in sequential operations aligns with the sequential character of DNA. Exemplary uses of RNNs to identify carrier statuses of sequenced genomes will now be described.

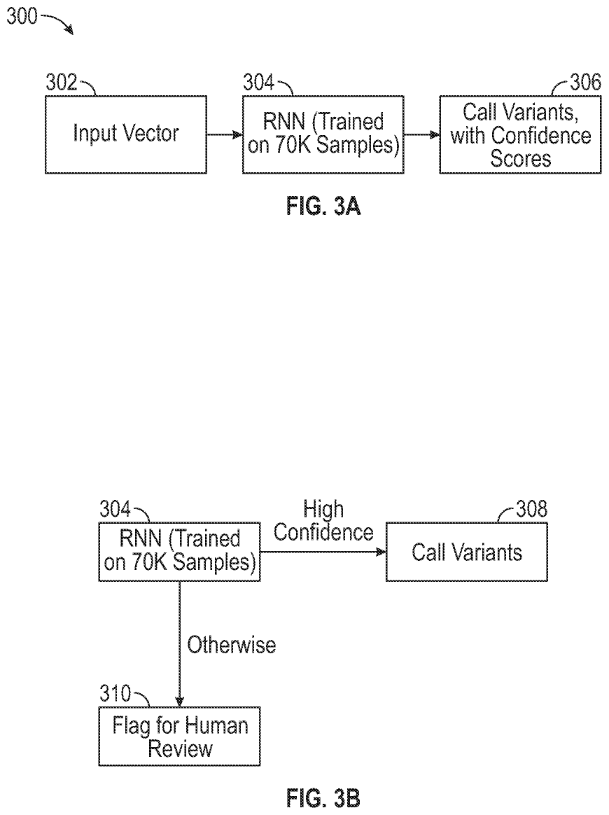

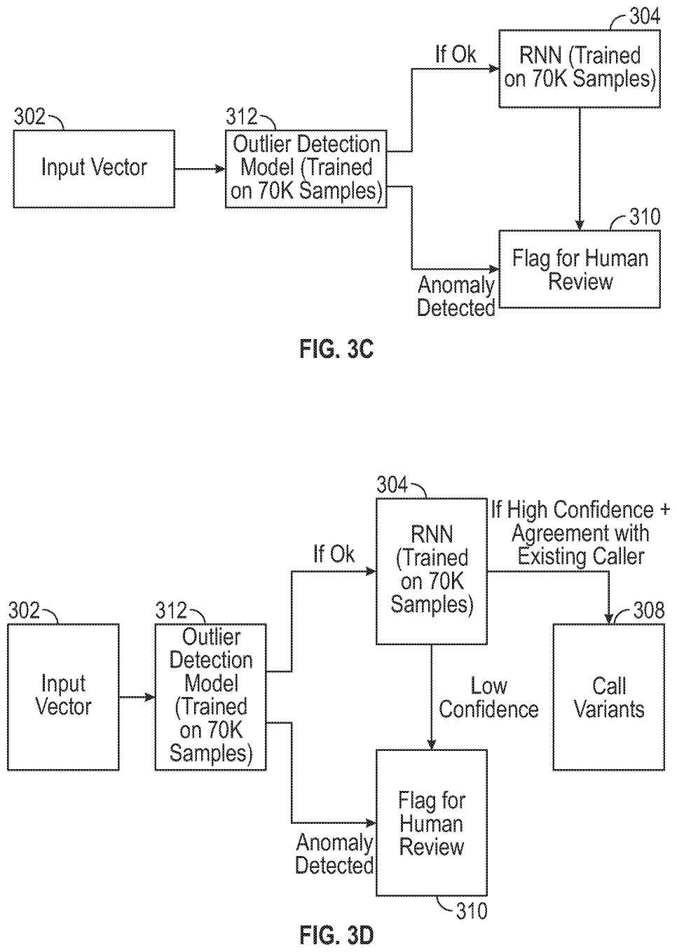

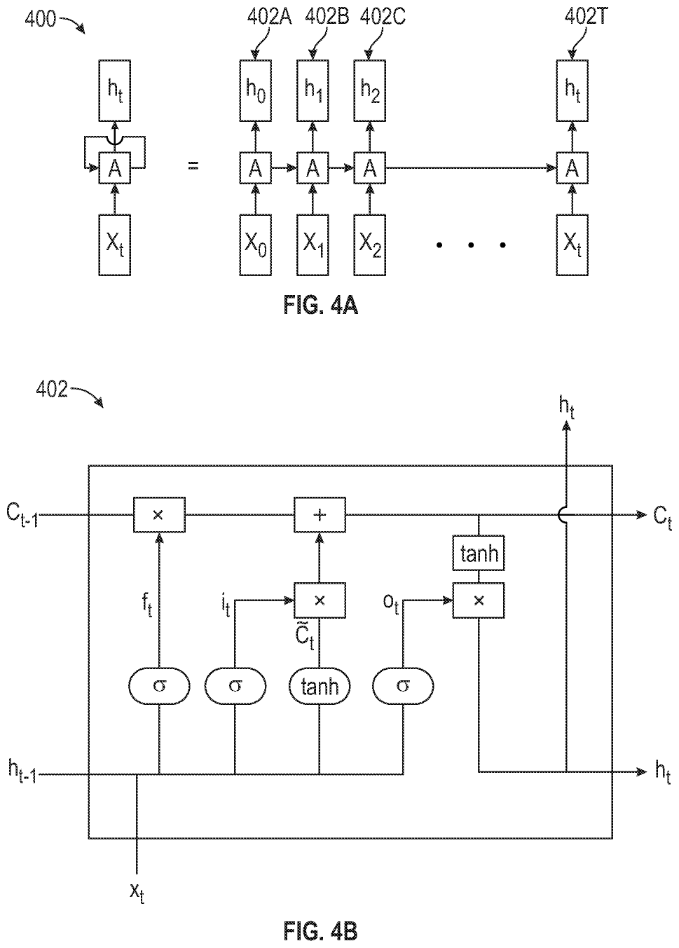

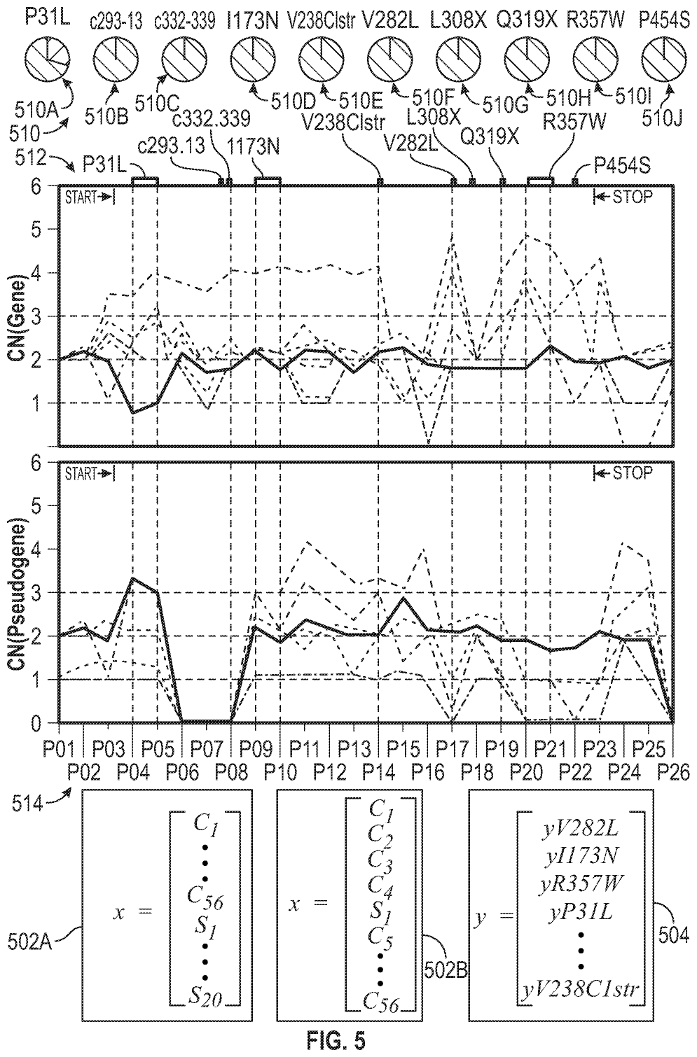

FIG. 3A illustrates an exemplary process 300 in which an RNN can be used to determine one or more carrier statuses associated with the genome (or portion of the genome) being sequenced according to examples of the disclosure. Input vector 302 can correspond to SNP and/or copy number data for that genome, as described above. Input vector 302 can be inputted to RNN 304 that has been trained with SNP and copy number data, and corresponding carrier status determinations, to output variant calls and/or confidence scores at 306. The SNP and copy number data, and the carrier status determinations, used to train RNN 304 can include SNP and copy number data from previously sequenced samples, and can include data from samples that are known carriers for the one or more genetic conditions RNN 304 is being trained to identify, data from samples that are known non-carriers for the one or more genetic conditions RNN 304 is being trained to identify, or a mixture of both known carriers and known non-carriers for the one or more genetic conditions RNN 304 is being trained to identify. Further, the known carrier statuses of those previously sequenced samples can be used to train RNN 304 to be able to connect SNP and copy number data with carrier statuses. Variant calls (provided in 306) in this context can be indications of whether or not the RNN 304 determines that the sample being sequenced is associated with one or more genetic conditions (e.g., CAH) or carrier statuses. Further, in some examples, RNN 304 can output, along with the variant calls themselves, indications of the confidence with which those variant calls are made (e.g., confidence scores ranging from 0 (least confident) to 1.0 (most confident), or activation values between 0 and 1 such that an activation value between 0 and 0.5 indicates that the sample is regarded as negative for the corresponding variant (0 being most-confidently negative, and 0.5 being least-confidently negative), and an activation value between 0.5 and 1 indicates that the sample is regarded as positive for the corresponding variant (1 being most-confidently positive, and 0.51 being least-confidently positive)). In such examples, the RNN 304 can be trained with confidence scores, in addition to the SNP, copy number and known carrier status determinations, so as to be able to produce confidence scores as outputs when used in process 300. Exemplary details of input vector 302, RNN 304 and variant calls 306 will be described later with reference to FIGS. 4A-4B and 5. Exemplary details of training data used to train RNN 304 will also be described later.

FIG. 3B illustrates an alternative flow for process 300 in which RNN 304 only outputs variant calls 308 if those calls are associated with relatively high confidence levels (e.g., confidence levels greater than a threshold confidence level, such as 0.8, 0.9 or 1.0 in the case of a statistical confidence model) according to examples of the disclosure. Specifically, if RNN 304 is able to produce variant calls 308 at such a high confidence level, then it outputs those variant calls at 308 (e.g., inserted into a patient report to be sent to the patient with minimal additional review). However, if RNN 304 is not able to produce variant calls 308 at such a high confidence level (e.g., the confidence level is less than or equal to the above threshold confidence level), then RNN 304 does not output variant calls 308; rather, the SNP data, copy number data, variant calls and/or confidence levels are flagged for review (e.g., flagged for detailed human review and/or put into a non-RNN-based variant calling and review process, without being inserted into a patient report) at 310.

In some examples, it can be beneficial to only input SNP and copy number data for a genome being sequenced to RNN 304 if that data is not considered to be outlier data (e.g., data in which one or more anomalies are detected). Anomalies in CYP21A2 might include noisy sequencing data or uncommon forms of genetic variation. FIG. 3C illustrates an exemplary process 301 in which anomaly detection is used before data is inputted to RNN 304 for determining one or more carrier statuses associated with the genome (or portion of the genome) being sequenced according to examples of the disclosure. Input vector 302 can be inputted to anomaly detection model 312. If anomaly detection model 312 determines that the data in input vector 302 is not anomalous, then that input vector 302 can be inputted to RNN 304. If, however, anomaly detection model 312 determines that the data in input vector 302 is anomalous, then the SNP data, copy number data, variant calls and/or confidence levels can be flagged for review (e.g., flagged for human review) at 310, without inputting vector 302 to RNN 304. In some examples, anomaly detection model 312 can determine that a given set of data is anomalous if it corresponds to one or more variant calls (e.g., carrier status determinations) that required human review and/or override, because a calling algorithm (e.g., carrier status determination algorithm) that is not based on the machine learning algorithms of the disclosure (e.g., one used for production samples, such as a variant calling algorithm that uses base counting and a log-odds ratio threshold to classify variants, or a variant calling algorithm based on manual review of the sequencing data) was not able to produce a confident call (e.g., carrier status determination), or produced an inaccurate call (e.g., carrier status determination). In some embodiments, anomaly detection model 312 comprises a machine learning algorithm (e.g., a support vector machine) that is trained to predict whether a sample will be “overridden” in call review. For example, given inputs of the same SNP data and copy number data, the anomaly detection model 312 can learn to predict whether a sample is likely to be “overridden”, and is thus anomalous.

FIG. 3D illustrates another exemplary process 303 for determining one or more carrier statuses associated with the genome being sequenced in which anomaly detection and flagging for review are utilized according to examples of the disclosure. Input vector 302 can be inputted to anomaly detection model 312. If anomaly detection model 312 determines that the data in input vector 302 is not anomalous (e.g., as described with reference to 312 in FIG. 3C), then that input vector 302 can be inputted to RNN 304. If, however, anomaly detection model 312 determines that the data in input vector 302 is anomalous, then the SNP data, copy number data, variant calls and/or confidence levels can be flagged for review (e.g., flagged for human review) at 310, without inputting vector 302 to RNN 304 (e.g., as described with reference to 310 in FIG. 3C).

If RNN 304 is able to produce variant calls 308 with relatively high confidence levels (e.g., confidence levels greater than a threshold confidence level, such as 0.8, 0.9 or 1.0 on the above-described scale from 0 to 1), then it can output those variant calls at 308. In some examples, RNN 304 may be required to produce variant calls 308 at the above relatively high confidence level, and those variant calls may be required to be in agreement with another variant calling algorithm (a non-RNN-based variant caller, or a variant caller other than the RNN-based caller described here, such as a variant calling algorithm that uses base counting and a log-odds ratio threshold to classify variants, or a variant calling algorithm based on manual review of the sequencing data) in order for RNN 304 to output those variant calls at 308. However, if RNN 304 is not able to produce variant calls 308 at such a high confidence level (e.g., the confidence level is less than or equal to the above threshold confidence level and/or the variant calls produced by RNN 304 are not in agreement with the other variant calling algorithm), then RNN 304 does not output variant calls 308; rather, the SNP data, copy number data, variant calls and/or confidence levels are flagged for review (e.g., flagged for human review) at 310 (e.g., as described with reference to 310 in FIG. 3C).

As previously mentioned, various machine learning algorithms and/or architectures can be utilized in making carrier status determinations based on SNP and copy number data according to the examples of the disclosure. In some examples, RNNs can be utilized. FIGS. 4A-4B illustrate exemplary details of a RNN that can be utilized for determining one or more carrier statuses associated with the genome (or portion of the genome) being sequenced according to examples of the disclosure. For example, as shown in FIG. 4A, RNN 400 having input(s) Xₜ, output(s) hₜ and transition function F can be utilized. Input(s) Xₜ can be the SNP and copy number data of the genome being sequenced, and output(s) hₜ can be the determined carrier statuses of the genome being sequenced—exemplary details of input(s) Xₜ and output(s) hₜ will be described with reference to FIG. 5. When unrolled or unfolded, RNN 400 can be represented by layers 402A, 402B, etc., where layer 402A can have input X₀ (e.g., first SNP or copy number data value) and output h₀, layer 402B can have input X₁ (e.g., second SNP or copy number data value) and output h₁, etc. RNN 400 can be structured to receive, as inputs, input vector 302, and output call variants and/or confidence scores 306 or 308, exemplary details of which will be described with reference to FIG. 5. In some examples, an RNN of the disclosure can have different structure depending on the form of the input vector and the output call variants, which can be different for different genetic conditions to be determined (e.g., different numbers of copy number data points, different numbers of SNP data points due to different numbers of known SNPs that contribute to the different genetic conditions, different numbers of carrier statuses to be determined due to different numbers of carrier statuses associated with different genetic conditions, SNP data relating to a particular variant location separated by probe (e.g., as compared with aggregated SNP data from multiple probes for a particular variant location), etc.)—such RNNs can be structured analogously to those described here.

FIG. 4B illustrates exemplary details of a given layer of RNN 400 according to examples of the disclosure. The structure of FIG. 4B can be the structure of each of layers 402A, 402B, etc., which can be structured as long-short term memory (LS™) cells. Each cell can operate according to the following equations:

fₜ=σ(W_{f}·[hₜ₋₁,xₜ]+b_{f})

iₜ=σ(Wᵢ·[hₜ₋₁,xₜ]+bᵢ)

\tilde{C}ₜ=tanh(W_{C}·[hₜ₋₁,xₜ]+b_{C})

Cₜ=fₜ*Cₜ₋₁+iₜ*Cᵣ=state of cell/layer t

σₜ=σ(Wₒ·[hₜ₋₁,xₜ]+bₒ)

hₜ=oₜ*tanh(Cₜ)

where xₜ can be the input vector for the LSTM cell, fₜ can be the forget gate's activation function, iₜ can be the input gate's activation function, oₜ can be the output gate's activation function, hₜ can be the output vector of the LSTM cell, W and b can be weight matrix and bias vector parameters that can be learned during training, σ can be a Sigmoid function, and * can be a Hadamard (entry-wise) product.

Because genomic samples that are not carriers for one or more genetic conditions, such as CAH, can far outnumber genomic samples that are carriers for one or more genetic conditions (e.g., because genetic conditions can be relatively rare), the data on which RNN 400 can be trained and/or to which RNN 400 can be applied can have a relatively large class imbalance between negative samples (e.g., genomic samples that are not carriers for one or more genetic conditions) and positive samples (e.g., genomic samples that are carriers for one or more genetic conditions). As such, it can be beneficial to utilize weighted cross-entropy loss functions in the RNN-based processes of the disclosure to up-weight the significance of positive samples on RNN operation when training the RNN. One exemplary weighted cross-entropy loss function can be expressed as:

$$L = - \frac{1}{N} {\sum }_{i = 1}^{N} {\sum }_{j = 1}^{M} [ {C}_{j} {y}_{ij} log {\hat{y}}_{ij} + ( 1 - {y}_{ij} ) log ( 1 - {\hat{y}}_{ij} )$$

where yᵢⱼ can be the carrier status for a given patient (sample) i and variant j (e.g., if patient (sample) i is a carrier for variant j, yᵢⱼ=1, and if patient (sample) i is not a carrier for variant j, yᵢⱼ=0), ŷᵢⱼ can be the probability of finding yᵢⱼ=1, and C₃ can be expressed as:

$$\begin{matrix}{C}_{j} = positive oversampling coefficient for variant j \\ = \frac{number of negative examples , variant j}{number of positive samples , variant j}\end{matrix}$$

A loss function (e.g., the weighted cross-entropy loss function above) can be a metric that measures how well the predictions of the variant callers of the disclosure agree with the provided training data (e.g., higher is worse agreement, lower is better agreement). In some examples, the RNN parameters can be varied so as to gradually decrease this loss function so as to train the RNN, as described in this disclosure. In the specific loss function shown above, the average cross-entropy loss over all N samples in the relevant set (e.g., the size of the training set). Further, the respective losses over each of the M variants of interest can be summed (e.g., 11 variants in the case of one of the CAH callers of the disclosure).

The SNP, copy number and carrier status (“variant call”) data used to train the RNNs of the disclosure and used during the operation of the RNNs of the disclosure to determine carrier status can be represented in any suitable manner, though some ways of representing the above data can result in better RNN performance (e.g., more accurate carrier status determinations, faster carrier status determinations, etc.) than others. FIG. 5 illustrates exemplary structures of SNP, copy number and carrier status data according to examples of the disclosure. SNP data 510 and copy number data 512 and 514 can be as described with reference to FIG. 2B for a gene-pseudogene pair of interest. Such data for use in the RNNs of the disclosure can be represented as illustrated in FIG. 5. Specifically, in some examples, carrier status determinations can be represented as one-dimensional array y 504 having one entry for each carrier status to be determined (in the case of using the RNNs of the disclosure to determine carrier status from SNP and copy number data) or one entry for each known carrier status (in the case of training the RNNs of the disclosure using SNP and copy number data for known carrier statuses). For example, for the purposes of using the RNNs of the disclosure in the context of CYP21A2 and CYP21A1P for CAH, array y 504 can include 10 entries corresponding to P31L, c293-13, c332-339, I173N, V238Clstr, V282L, L308X, Q319X, R357W and P454S carriers (and in some examples, an entry corresponding to the 30-kb deletion as well). It is understood that additional or alternative variants (and variant-entries) can be utilized. In the context of other gene-pseudogene pairs of interest, array y can include fewer or more entries, each corresponding to a carrier status of interest. In some examples, array y can include more than one entry per carrier status—for example, to be able to separately provide carrier status/variant determinations on a per-chromosome or per-gene basis. For example, if the carrier status of interest is one that can show up separately in each chromosome of the individual, array y can be twice the length of the above examples (i.e., array y can include two entries per carrier status: one for the carrier status in the first chromosome of the individual, and one for the carrier status in the second chromosome of the individual) to separately indicate the existence or non-existence of the variant of interest in each of the first and second chromosomes of the individual. For some genes, array y may need to be arbitrarily increased in length to add additional entries for a given carrier status, because some patients may have more than two copies of the gene of interest (e.g., in the case of CAH, more than two copies of CYP21A2), and thus array y can include sufficient entries for a given carrier status to correspond to each of the more than two copies of the gene of interest.

In some examples, the values for each entry in array y 504 can be binary (e.g., 0 for non-carrier, and 1 for carrier). In some examples, the values for each entry can indicate the confidence with which such carrier status is expressed/determined (e.g., 0 for 100% confident non-carrier, 1 for 100% confident carrier, and decimal values between 0 and 1 corresponding to different non-carrier or carrier confidence levels). In some examples, the values for each entry in array y 504 can be binary for training purposes, and can indicate the confidence with which such carrier status is expressed/determined when the RNN is being used to determine variant calls. The ordering of the entries in array y 504 can be varied. Because RNNs can be especially effective in the context of sequential data, the performance of the RNN-based processes of the disclosure can be improved by representing the carrier status data in array y 504 in a manner having a sequential characteristic that corresponds to the sequence of the genetic material in the gene/pseudogene of interest. For example, in some examples, the ordering of the entries in array y 504 can correspond to the positioning of the mutations in the gene/pseudogene of interest associated with each carrier status. For example, an entry for a carrier status that is associated with a mutation closest to the 5′ end of the gene/pseudogene of interest can be located at the first position in array y 504, an entry for a carrier status that is associated with a mutation closest to the 3′ end of the gene/pseudogene of interest can be located at the last position in array y 504, and entries for carrier statuses that are associated with mutations at other positions in the gene/pseudogene can be located at other corresponding positions in array y 504. In some examples, the ordering of the carrier status entries in array y 504 may not correspond to the positioning of the mutations in the gene/pseudogene of interest associated with each carrier status, and may be independent of such positioning.

In some examples, SNP and copy number data can be combined into a single one-dimensional input array x. The ordering of the entries in array x can be varied. For example, in array x 502A, copy number and SNP data can be arranged such that copy number data from the 5′ end of the gene of interest to the 3′ end of the gene of interest can be located in the first part of array x 502A (e.g., the first 28 entries of array x 502A in the case where copy number data from 28 positions across the gene is available), copy number data from the 5′ end of the corresponding pseudogene to the 3′ end of the corresponding pseudogene can be located in the second part of array x 502A (e.g., the second 28 entries of array x 502A in the case where copy number data from 28 positions across the pseudo gene is available), and SNP data from the 5′ end of the gene and/or pseudogene to the 3′ end of the gene and/or pseudogene can be located in the third part of array x 502A (e.g., the last 20 entries of array x 502A in the case where SNP data from 10 positions across the gene is available, and SNP data from 10 positions across the pseudogene is available, or the last 10 entries of array x 502A in the case where SNP data from 10 positions across the gene is available but no SNP data from the pseudogene is available or utilized). For example, the contents and order of array x can be expressed as:

x=[CN_{gene,i},CN_{gene,i+1},CN_{gene,i+2}, . . . ,CN_{pseudogene,i},CN_{pseudogene,i+1},CN_{pseudogene,i+2}, . . . ,SNP_{gene,i},SNP_{gene,i+1},SNP_{gene,i+2}, . . . ,SNP_{pseudogene,i},SNP_{pseudogene,i+1},SNP_{pseudogene,i+2}, . . . ]

where CN_{gene,i} can be the copy number data for the gene at position i, SNP_{gene,i} can be the SNP data for the gene at position i, CN_{pseudogene,i} can be the copy number data for the pseudogene at position i, and SNP_{pseudogene,i} can be the SNP data for the gene at position i. If no copy number or SNP data exists for a given position in the gene or pseudogene, the corresponding entry in array x can be omitted. The above arrangement of the SNP and copy number data is illustrated in array x 502A of FIG. 5, where C₁ to C₅₆ correspond to the 56 entries of copy number data described above, and S₁ to S₂₀ correspond to the 20 entries of SNP data described above.

Because RNNs can be especially effective in the context of sequential data, the performance of the RNN-based processes of the disclosure can be improved by representing the SNP and copy number data in a manner have a sequential characteristic that corresponds to the sequence of the genetic material in the gene/pseudogene of interest. For example, SNP and copy number data can be organized in array x such that the order in which the SNP and copy number data appears in array x corresponds to the location in the gene/pseudogene to which the SNP and copy number data corresponds. More specifically, SNP and copy number data corresponding to a position closest to the 5′ end of the gene/pseudogene can be located at the front end of array x, SNP and copy number data corresponding to a position closest to the 3′ end of the gene/pseudogene can be located at the back end of array x, and SNP and copy number data corresponding to other positions in the gene/pseudogene can be located at other corresponding positions in array x. For example, the contents and order of array x can be expressed as:

x=[CN_{gene,i},SNP_{gene,i},CN_{pseudogene,i},SNP_{pseudogene,i},CN_{gene,i+1},SNP_{gene,i+1},CN_{pseudogene,i+1},SNP_{pseudogene,i+1}, . . . ],

x=[CN_{gene,i},CN_{pseudogene,i},SNP_{gene,i},CN_{gene,i+1},CN_{pseudogene,i+1},SNP_{gene,i+1},CN_{gene,i+2},CN_{pseudogene,i+2},SNP_{gene,i+2}, . . . ], or

x=[CN_{gene,i},CN_{pseudogene,i},SNP_{gene,i},SNP_{pseudogene,i},CN_{gene,i+1},CN_{pseudogene,i+1},SNP_{gene,i+1},SNP_{pseudogene,i+1}, . . . ]

where CN_{gene,i} can be the copy number data for the gene at position i, SNP_{gene,i} can be the SNP data for the gene at position i, CN_{pseudogene,i} can be the copy number data for the pseudogene at position i, and SNP_{pseudogene,i} can be the SNP data for the gene at position i. If no copy number or SNP data exists for a given position in the gene or pseudogene, the corresponding entry in array x can be omitted. The above arrangement of the SNP and copy number data is illustrated in array x 502B of FIG. 5.

Other arrangements of SNP and copy number data in array x are also within the scope of the disclosure. Below are some additional exemplary arrangements for such data, some of which have a partial or full sequential characteristic that corresponds to the sequence of the genetic material in the gene/pseudogene of interest:

x=[SNP_{gene,i},SNP_{pseudogene,i},SNP_{gene,i+1},SNP_{pseudogene,i+1}, . . . ,CN_{gene,i},CN_{pseudogene,i},CN_{gene,i+1},CN_{pseudogene,i+1}, . . . ]

x=[SNP_{gene,i},SNP_{gene,i+1}, . . . ,SNP_{pseudogene,i},SNP_{pseudogene,i+1}, . . . ,CN_{gene,i},CN_{gene,i+1}, . . . ,CN_{pseudogene,i},CN_{pseudogene,i+1}, . . . ]

While the data above was discussed in the context of arrays, it is understood that other data structures (e.g., matrices, lists, etc.)—some of which that can be used to convey ordering of their entries (e.g., an ordering characteristic that can convey a “first” position, a “last” position, and/or relative positions of entries within the data structure, etc.), and some of which that do not convey ordering of their entries—can additionally or alternatively be used to represent the copy number data, the SNP data and/or the carrier status determinations. While the examples of the disclosure have been described with the RNN determining carrier statuses of the individual, it is understood that the RNN can be analogously configured to additionally or alternatively determine the number of functional copies of a given gene in the individual's genome (which is related to the carrier statuses described above). In such examples, the output data from the RNN (e.g., during training and/or during use) can include the number of functional copies of a given gene additionally or alternatively to the carrier statuses of the individual.

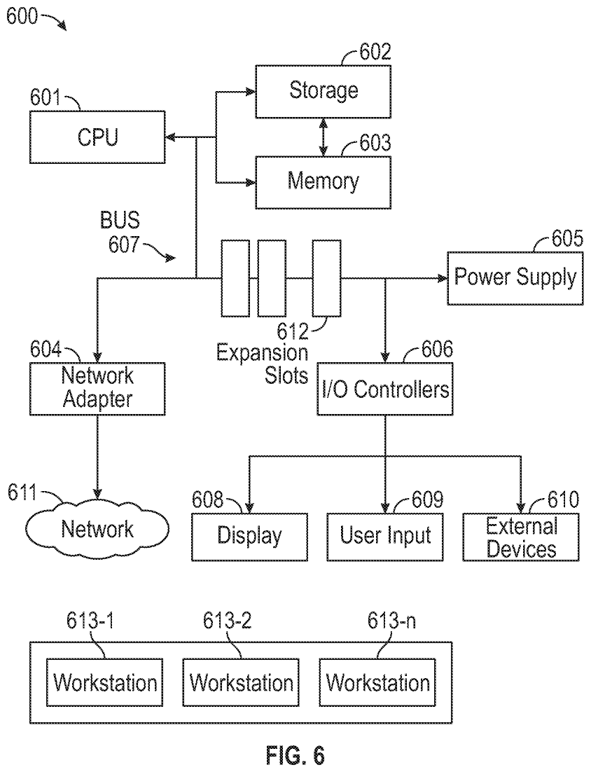

FIG. 6 illustrates an exemplary computing system or electronic device for implementing the examples of the disclosure. System 600 may include, but is not limited to known components such as central processing unit (CPU) 601, storage 602, memory 603, network adapter 604, power supply 605, input/output (I/O) controllers 606, electrical bus 607, one or more displays 608, one or more user input devices 609, and other external devices 610. It will be understood by those skilled in the art that system 600 may contain other well-known components which may be added, for example, via expansion slots 612, or by any other method known to those skilled in the art. Such components may include, but are not limited, to hardware redundancy components (e.g., dual power supplies or data backup units), cooling components (e.g., fans or water-based cooling systems), additional memory and processing hardware, and the like.

System 600 may be, for example, in the form of a client-server computer capable of connecting to and/or facilitating the operation of a plurality of workstations or similar computer systems over a network. In another embodiment, system 600 may connect to one or more workstations over an intranet or internet network, and thus facilitate communication with a larger number of workstations or similar computer systems. Even further, system 600 may include, for example, a main workstation or main general purpose computer to permit a user to interact directly with a central server. Alternatively, the user may interact with system 600 via one or more remote or local workstations 613. As will be appreciated by one of ordinary skill in the art, there may be any practical number of remote workstations for communicating with system 600.

CPU 601 may include one or more processors, for example Intel® Core™ i7 processors, AMD FX™ Series processors, or other processors as will be understood by those skilled in the art (e.g., including graphical processing unit (GPU)-style specialized computing hardware used for, among other things, machine learning applications, such as training and/or running the machine learning algorithms of the disclosure; such GPUs may include, e.g., NVIDIA Tesla™ K80 processors). CPU 601 may further communicate with an operating system, such as Windows NT® operating system by Microsoft Corporation, Linux operating system, or a Unix-like operating system. However, one of ordinary skill in the art will appreciate that similar operating systems may also be utilized. Storage 602 (e.g., non-transitory computer readable medium) may include one or more types of storage, as is known to one of ordinary skill in the art, such as a hard disk drive (HDD), solid state drive (SSD), hybrid drives, and the like. In one example, storage 602 is utilized to persistently retain data for long-term storage. Memory 603 (e.g., non-transitory computer readable medium) may include one or more types of memory as is known to one of ordinary skill in the art, such as random access memory (RAM), read-only memory (ROM), hard disk or tape, optical memory, or removable hard disk drive. Memory 603 may be utilized for short-term memory access, such as, for example, loading software applications or handling temporary system processes.

As will be appreciated by one of ordinary skill in the art, storage 602 and/or memory 603 may store one or more computer software programs. Such computer software programs may include logic, code, and/or other instructions to enable processor 601 to perform the tasks, operations, and other functions as described herein (e.g., the RNN functions described herein), and additional tasks and functions as would be appreciated by one of ordinary skill in the art. Operating system 602 may further function in cooperation with firmware, as is well known in the art, to enable processor 601 to coordinate and execute various functions and computer software programs as described herein. Such firmware may reside within storage 602 and/or memory 603.

Moreover, I/O controllers 606 may include one or more devices for receiving, transmitting, processing, and/or interpreting information from an external source, as is known by one of ordinary skill in the art. In one embodiment, I/O controllers 606 may include functionality to facilitate connection to one or more user devices 609, such as one or more keyboards, mice, microphones, trackpads, touchpads, or the like. For example, I/O controllers 606 may include a serial bus controller, universal serial bus (USB) controller, FireWire controller, and the like, for connection to any appropriate user device. I/O controllers 606 may also permit communication with one or more wireless devices via technology such as, for example, near-field communication (NFC) or Bluetooth™. In one embodiment, I/O controllers 606 may include circuitry or other functionality for connection to other external devices 610 such as modem cards, network interface cards, sound cards, printing devices, external display devices, or the like. Furthermore, I/O controllers 606 may include controllers for a variety of display devices 608 known to those of ordinary skill in the art. Such display devices may convey information visually to a user or users in the form of pixels, and such pixels may be logically arranged on a display device in order to permit a user to perceive information rendered on the display device. Such display devices may be in the form of a touch-screen device, traditional non-touch screen display device, or any other form of display device as will be appreciated be one of ordinary skill in the art.

Furthermore, CPU 601 may further communicate with I/O controllers 606 for rendering a graphical user interface (GUI) on, for example, one or more display devices 608. In one example, CPU 601 may access storage 602 and/or memory 603 to execute one or more software programs and/or components to allow a user to interact with the system as described herein. In one embodiment, a GUI as described herein includes one or more icons or other graphical elements with which a user may interact and perform various functions. For example, GUI 607 may be displayed on a touch screen display device 608, whereby the user interacts with the GUI via the touch screen by physically contacting the screen with, for example, the user's fingers. As another example, GUI may be displayed on a traditional non-touch display, whereby the user interacts with the GUI via keyboard, mouse, and other conventional I/O components 609. GUI may reside in storage 602 and/or memory 603, at least in part as a set of software instructions, as will be appreciated by one of ordinary skill in the art. Moreover, the GUI is not limited to the methods of interaction as described above, as one of ordinary skill in the art may appreciate any variety of means for interacting with a GUI, such as voice-based or other disability-based methods of interaction with a computing system.

Moreover, network adapter 604 may permit device 600 to communicate with network 611. Network adapter 604 may be a network interface controller, such as a network adapter, network interface card, LAN adapter, or the like. As will be appreciated by one of ordinary skill in the art, network adapter 604 may permit communication with one or more networks 611, such as, for example, a local area network (LAN), metropolitan area network (MAN), wide area network (WAN), cloud network (IAN), or the Internet.

One or more workstations 613 may include, for example, known components such as a CPU, storage, memory, network adapter, power supply, I/O controllers, electrical bus, one or more displays, one or more user input devices, and other external devices. Such components may be the same, similar, or comparable to those described with respect to system 600 above. It will be understood by those skilled in the art that one or more workstations 613 may contain other well-known components, including but not limited to hardware redundancy components, cooling components, additional memory/processing hardware, and the like.

### EXAMPLES

### Example 1: RNN Trained on 76,723 Samples Using Structure of Array X 502A

A RNN was constructed using the TensorFlow software library. In particular, using the Python API, a symbolic computation graph was constructed that executes in the TensorFlow runtime. The TensorFlow RNN was constructed of 5 layers of LSTM cells with 11 output nodes, the operations of which were described with reference to FIGS. 4A-4B. SNP, copy number and known carrier status data from 76,723 previously-sequenced genome samples (a mixture of CAH positive and negative samples, with approximately 8% of the samples being positive) were formatted into arrays x and y having structures illustrated in FIG. 5 (e.g., array x 502A, array y 504), and stored as NumPy arrays in the HDF5 data model, library, and file format. Those arrays corresponding to 80% of the previously-sequenced 95,903 samples were used to train the RNN constructed in TensorFlow.

After training the RNN, the arrays corresponding to the remaining 20% of the previously-sequenced 95,903 samples were used to test the performance of the trained RNN. The trained RNN produced carrier status determinations that were 99.89% accurate, with a 1 in 900 error rate. Various sensitivities and specificities based on specific carrier statuses were observed, as shown in the table below. In some examples, “sensitivity” is defined as TP/(TP+FN), where TP can be the number of true positives for a variant, and FN can be the number of false negatives for a variant. In some examples, “specificity” can be defined as TN/(TN+FP), where TN can be the number of true negatives for a variant, and FP can be the number of false positives for a variant.

| | | |
|---|---|---|
| Variant | Sensitivity | Specificity |

| | | | | |
|---|---|---|---|---|
| V238Clstr | 100 | (9/9) | 99.99 | (19169/19171) |
| 30kb_del | 99.65 | (1125/1129) | 99.98 | (18048/18051) |
| L308X | 100 | (2/2) | 100 | (19178/19178) |
| Q319X | 99.77 | (427/428) | 100 | (18752/18752) |
| c332-339 | 83.33 | (5/6) | 100 | (19174/19174) |
| P454S | 100 | (226/226) | 100 | (18954/18954) |
| c293-13 | 98.94 | (93/94) | 100 | (19086/19086) |
| P31L | 100 | (13/13) | 100 | (19167/19167) |
| R357W | 88.89 | (8/9) | 99.99 | (19170/19171) |
| I173N | 97.66 | (125/128) | 99.99 | (19050/19052) |
| V282L | 100 | (763/763) | 99.99 | (18415/18417) |

Although examples of this disclosure have been fully described with reference to the accompanying drawings, it is to be noted that various changes and modifications will become apparent to those skilled in the art. Such changes and modifications are to be understood as being included within the scope of examples of this disclosure as defined by the appended claims.

1. A method for determining a respective carrier status of an individual, the method comprising:
determining the respective carrier status based on copy number data for a gene in a genome of the individual and SNP data for the gene using a machine learning algorithm, wherein the machine learning algorithm is configured to receive, as inputs, the copy number data and the SNP data, and output the respective carrier status of the individual.

2. The method of claim 1, wherein the gene is sequenced to determine the copy number data and the SNP data.

3. The method of claim 2, wherein the gene is sequenced using direct targeted sequencing to determine the copy number data and the SNP data.

4. The method of any one of claims 1-3, wherein the respective carrier status is associated with a genetic condition.

5. The method of claim 4, wherein:
the genetic condition is congenital adrenal hyperplasia (CAH), and
the respective carrier status comprises one or more of V238Clstr, 30kb_del, L308X, Q319X, c332-339, P454S, c293-13, P31L, R357W, I173N and V282L carrier statuses.

6. The method of any one of claims 1-5, wherein the genome comprises a gene associated with the respective carrier status and/or a pseudogene corresponding to the gene, and the copy number data is indicative of a number of copies of genetic material corresponding to the gene and/or the pseudogene that are detected during sequencing of the genome at a plurality of locations across the genome.

7. The method of any one of claims 1-6, wherein the SNP data is indicative of a number of sequencing reads from the gene that have a single nucleotide polymorphism relative to a reference sequence at one or more locations across the gene.

8. The method of any one of claims 1-7, wherein prior to determining the respective carrier status using the machine learning algorithm, the machine learning algorithm is trained using copy number data, SNP data and known carrier status data for one or more other genetic samples from other individuals.

9. The method of claim 8, wherein the one or more other genetic samples include one or more known carrier genetic samples and one or more known non-carrier genetic samples.

10. The method of any one of claims 1-9, further comprising determining a plurality of carrier statuses of the individual, including the respective carrier status, wherein the machine learning algorithm includes an output for each carrier status of the plurality of carrier statuses.

11. The method of any one of claims 1-10, wherein:
the copy number data comprises one or more copy number values,
the SNP data comprises one or more SNP values, and
the machine learning algorithm includes an input for:
each copy number value of the one or more copy number values, and
each SNP value of the one or more SNP values.

12. The method of claim 11, wherein each input of the machine learning algorithm associated with the one or more copy number values corresponds to a unique sequencing probe used to sequence the gene.

13. The method of any one of claims 11-12, wherein each input of the machine learning algorithm associated with the one or more SNP values corresponds to a location in the gene associated with one or more probes used to sequence the gene.

14. The method of any one of claims 1-13, wherein determining the respective carrier status comprises:
in accordance with a determination that the respective carrier status is determined with a confidence level below a confidence level threshold, flagging the determined respective carrier status for review; and
in accordance with a determination that the respective carrier status is determined with a confidence level above the confidence level threshold, forgoing flagging the determined respective carrier status for review.

15. The method of claim 14, wherein determining the respective carrier status with the confidence level above the confidence level threshold includes determining that the respective carrier status determined using the machine learning algorithm is in agreement with a carrier status determination for the individual from a carrier status determination algorithm different than the machine learning algorithm.

16. The method of any one of claims 1-15, further comprising:
prior to determining the respective carrier status of the individual, determining whether the copy number data or the SNP data are statistically anomalous; and
in accordance with a determination that the copy number data and/or the SNP data are statistically anomalous, flagging the copy number data and/or the SNP data for review.

17. The method of any one of claims 1-16, further comprising determining a confidence level for the determination of the respective carrier status using the machine learning algorithm, wherein the machine learning algorithm is further configured to output the confidence level.

18. The method of any one of claims 1-17, wherein the machine learning algorithm comprises a neural network configured to receive the copy number data and the SNP data as inputs, and output the respective carrier status of the individual.

19. The method of claim 18, wherein the neural network comprises a recurrent neural network.

20. The method of any one of claims 1-19, wherein the copy number data is represented as a data structure having an ordering characteristic, and the copy number data is ordered within the data structure in an order that corresponds to a sequence of genetic material in the gene.

21. The method of claim 20, wherein:
the copy number data includes a first copy number data value corresponding to a first position in the gene, and a second copy number data value corresponding to a second position in the gene, the first position in the genome having a relative position with respect to the second position in the gene, and
the first copy number data value has the relative position with respect to the second copy number data value in the data structure.

22. The method of any one of claims 20-21, wherein the copy number data for a given gene in the genome is ordered in an order that corresponds to the sequence of genetic material in the given gene in the genome.

23. The method of any one of claims 20-22, wherein the copy number data for a given pseudogene in the genome is ordered in an order that corresponds to the sequence of genetic material in the given pseudogene in the genome.

24. The method of any one of claims 20-23, wherein:
the copy number data for a given gene in the genome and the copy number data for a given pseudogene in the genome are ordered in an order that corresponds to the sequence of genetic material in the given gene and the given pseudogene in the genome.

25. The method of any one of claims 20-24, wherein the SNP data is represented in the data structure and corresponds to one or more positions in the genome, and is ordered within the data structure in an order that corresponds to an order of the one or more positions in the genome.

26. The method of claim 25, wherein:
the SNP data includes a first SNP data value corresponding to a first position in the genome,
the copy number data includes a first copy number data value corresponding to a second position in the genome,
the first position in the genome has a relative position with respect to the second position in the genome, and
the first SNP data value has the relative position with respect to the first copy number data value in the data structure.

27. The method of any one of claims 25-26, wherein the copy number data for a given gene in the genome, the copy number data for a given pseudogene in the genome, and the SNP data corresponding to the one or more positions in the genome are ordered in an order that corresponds to the sequence of genetic material in the given gene and the given pseudogene, and the one or more locations in the genome.

28. The method of any one of claims 1-19, wherein the SNP data is represented as a data structure having an ordering characteristic, and the SNP data is ordered within the data structure in an order that corresponds to a sequence of genetic material in the gene.

29. The method of claim 28, wherein:
the SNP data includes a first SNP data value corresponding to a first position in the gene, and a second SNP data value corresponding to a second position in the gene, the first position in the genome having a relative position with respect to the second position in the gene, and
the first SNP data value has the relative position with respect to the second SNP data value in the data structure.

30. The method of any one of claims 28-29, wherein the SNP data for a given gene in the genome is ordered in an order that corresponds to the sequence of genetic material in the given gene in the genome.

31. The method of any one of claims 28-30, wherein the SNP data for a given pseudogene in the genome is ordered in an order that corresponds to the sequence of genetic material in the given pseudogene in the genome.

32. The method of any one of claims 28-31, wherein:
the SNP data for a given gene in the genome and the SNP data for a given pseudogene in the genome are ordered in an order that corresponds to the sequence of genetic material in the given gene and the given pseudogene in the genome.

33. The method of any one of claims 28-32, wherein the copy number data is represented in the data structure and corresponds to one or more positions in the genome, and is ordered within the data structure in an order that corresponds to an order of the one or more positions in the genome.

34. The method of claim 33, wherein:
the SNP data includes a first SNP data value corresponding to a first position in the genome,
the copy number data includes a first copy number data value corresponding to a second position in the genome,
the first position in the genome has a relative position with respect to the second position in the genome, and
the first SNP data value has the relative position with respect to the first copy number data value in the data structure.

35. The method of any one of claims 33-34, wherein the copy number data for a given gene in the genome, the copy number data for a given pseudogene in the genome, and the SNP data corresponding to the one or more positions in the genome are ordered in an order that corresponds to the sequence of genetic material in the given gene and the given pseudogene, and the one or more locations in the genome.

36. The method of any one of claims 1-35, further comprising determining a plurality of carrier statuses of the individual, including the respective carrier status, wherein:
the plurality of carrier statuses is represented as a data structure having an ordering characteristic, and the plurality of carrier statuses is ordered within the data structure in an order that corresponds to a sequence of genetic material in the genome.

37. The method of claim 36, wherein:
the plurality of carrier statuses includes a first carrier status associated with a first position in the genome, and a second carrier status associated with a second position in the genome, the first position in the genome having a relative position with respect to the second position in the genome, and
the first carrier status has the relative position with respect to the second carrier status in the data structure.

38. The method of any one of claims 1-37, wherein the copy number data and the SNP data are represented as a single data structure inputted to the machine learning algorithm.

39. The method of any one of claims 1-38, wherein determining the respective carrier status of the individual is further based on copy number data for a pseudogene in the genome of the individual corresponding to the gene, and SNP data for the pseudogene, wherein the machine learning algorithm is configured to further receive, as inputs, the copy number data for the pseudogene and the SNP data for the pseudogene.

40. The method of claim 39, wherein the gene and the pseudogene are selected from the group consisting of CYP21A2/CYP21A1P, GBA/psGBA, PMS2/PMS2CL and SMN1/SMN2.

41. The method of any one of claims 1-40, wherein the machine learning algorithm is trained on carrier statuses determined by human analysis (call review).

42. The method of any one of claims 1-41, wherein the machine learning algorithm is trained on carrier statuses determined by orthogonal experimental measurements.

43. The method of claim 42, wherein the orthogonal experimental measurements are long-range PCR and sequencing.

44. A method for determining a number of functional copies of a gene within a genome of an individual, the method comprising:
determining the number of functional copies of the gene based on copy number data for the gene and SNP data for the gene using a machine learning algorithm, wherein the machine learning algorithm is configured to receive, as inputs, the copy number data and the SNP data, and output the number of functional copies of the gene.

45. The method of claim 44, further comprising any one of the methods of claims 1-43.

46. A non-transitory computer readable storage medium storing instructions, which when executed by one or more processors of an electronic device, cause the electronic device to perform a method for determining a respective carrier status of an individual, the method comprising:
determining the respective carrier status based on copy number data for a gene in a genome of the individual and SNP data for the gene using a machine learning algorithm, wherein the machine learning algorithm is configured to receive, as inputs, the copy number data and the SNP data, and output the respective carrier status of the individual.

47. The non-transitory computer readable storage medium of claim 46, wherein the gene is sequenced to determine the copy number data and the SNP data.

48. The non-transitory computer readable storage medium of claim 47, wherein the gene is sequenced using direct targeted sequencing to determine the copy number data and the SNP data.

49. The non-transitory computer readable storage medium of any one of claims 46-48, wherein the respective carrier status is associated with a genetic condition.

50. The non-transitory computer readable storage medium of claim 49, wherein:
the genetic condition is congenital adrenal hyperplasia (CAH), and
the respective carrier status comprises one or more of V238Clstr, 30kb_del, L308X, Q319X, c332-339, P454S, c293-13, P31L, R357W, I173N and V282L carrier statuses.

51. The non-transitory computer readable storage medium of any one of claims 46-50, wherein the genome comprises a gene associated with the respective carrier status and/or a pseudogene corresponding to the gene, and the copy number data is indicative of a number of copies of genetic material corresponding to the gene and/or the pseudogene that are detected during sequencing of the genome at a plurality of locations across the genome.

52. The non-transitory computer readable storage medium of any one of claims 46-51, wherein the SNP data is indicative of a number of sequencing reads from the gene that have a single nucleotide polymorphism relative to a reference sequence at one or more locations across the gene.

53. The non-transitory computer readable storage medium of any one of claims 46-52, wherein prior to determining the respective carrier status using the machine learning algorithm, the machine learning algorithm is trained using copy number data, SNP data and known carrier status data for one or more other genetic samples from other individuals.

54. The non-transitory computer readable storage medium of claim 53, wherein the one or more other genetic samples include one or more known carrier genetic samples and one or more known non-carrier genetic samples.

55. The non-transitory computer readable storage medium of any one of claims 46-54, the method further comprising determining a plurality of carrier statuses of the individual, including the respective carrier status, wherein the machine learning algorithm includes an output for each carrier status of the plurality of carrier statuses.

56. The non-transitory computer readable storage medium of any one of claims 46-55, wherein:
the copy number data comprises one or more copy number values,
the SNP data comprises one or more SNP values, and
the machine learning algorithm includes an input for:
each copy number value of the one or more copy number values, and
each SNP value of the one or more SNP values.

57. The non-transitory computer readable storage medium of claim 56, wherein each input of the machine learning algorithm associated with the one or more copy number values corresponds to a unique sequencing probe used to sequence the gene.

58. The non-transitory computer readable storage medium of any one of claims 56-57, wherein each input of the machine learning algorithm associated with the one or more SNP values corresponds to a location in the gene associated with one or more probes used to sequence the gene.

59. The non-transitory computer readable storage medium of any one of claims 46-58, wherein determining the respective carrier status comprises:
in accordance with a determination that the respective carrier status is determined with a confidence level below a confidence level threshold, flagging the determined respective carrier status for review; and
in accordance with a determination that the respective carrier status is determined with a confidence level above the confidence level threshold, forgoing flagging the determined respective carrier status for review.

60. The non-transitory computer readable storage medium of claim 59, wherein determining the respective carrier status with the confidence level above the confidence level threshold includes determining that the respective carrier status determined using the machine learning algorithm is in agreement with a carrier status determination for the individual from a carrier status determination algorithm different than the machine learning algorithm.

61. The non-transitory computer readable storage medium of any one of claims 46-60, the method further comprising:
prior to determining the respective carrier status of the individual, determining whether the copy number data or the SNP data are statistically anomalous; and
in accordance with a determination that the copy number data and/or the SNP data are statistically anomalous, flagging the copy number data and/or the SNP data for review.

62. The non-transitory computer readable storage medium of any one of claims 46-61, the method further comprising determining a confidence level for the determination of the respective carrier status using the machine learning algorithm, wherein the machine learning algorithm is further configured to output the confidence level.

63. The non-transitory computer readable storage medium of any one of claims 46-62, wherein the machine learning algorithm comprises a neural network configured to receive the copy number data and the SNP data as inputs, and output the respective carrier status of the individual.

64. The non-transitory computer readable storage medium of claim 63, wherein the neural network comprises a recurrent neural network.

65. The non-transitory computer readable storage medium of any one of claims 46-64, wherein the copy number data is represented as a data structure having an ordering characteristic, and the copy number data is ordered within the data structure in an order that corresponds to a sequence of genetic material in the gene.

66. The non-transitory computer readable storage medium of claim 65, wherein:
the copy number data includes a first copy number data value corresponding to a first position in the gene, and a second copy number data value corresponding to a second position in the gene, the first position in the genome having a relative position with respect to the second position in the gene, and
the first copy number data value has the relative position with respect to the second copy number data value in the data structure.

67. The non-transitory computer readable storage medium of any one of claims 65-66, wherein the copy number data for a given gene in the genome is ordered in an order that corresponds to the sequence of genetic material in the given gene in the genome.

68. The non-transitory computer readable storage medium of any one of claims 65-67, wherein the copy number data for a given pseudogene in the genome is ordered in an order that corresponds to the sequence of genetic material in the given pseudogene in the genome.

69. The non-transitory computer readable storage medium of any one of claims 65-68, wherein:
the copy number data for a given gene in the genome and the copy number data for a given pseudogene in the genome are ordered in an order that corresponds to the sequence of genetic material in the given gene and the given pseudogene in the genome.

70. The non-transitory computer readable storage medium of any one of claims 65-69, wherein the SNP data is represented in the data structure and corresponds to one or more positions in the genome, and is ordered within the data structure in an order that corresponds to an order of the one or more positions in the genome.

71. The non-transitory computer readable storage medium of claim 70, wherein:
the SNP data includes a first SNP data value corresponding to a first position in the genome,
the copy number data includes a first copy number data value corresponding to a second position in the genome,
the first position in the genome has a relative position with respect to the second position in the genome, and
the first SNP data value has the relative position with respect to the first copy number data value in the data structure.

72. The non-transitory computer readable storage medium of any one of claims 70-71, wherein the copy number data for a given gene in the genome, the copy number data for a given pseudogene in the genome, and the SNP data corresponding to the one or more positions in the genome are ordered in an order that corresponds to the sequence of genetic material in the given gene and the given pseudogene, and the one or more locations in the genome.

73. The non-transitory computer readable storage medium of any one of claims 46-64, wherein the SNP data is represented as a data structure having an ordering characteristic, and the SNP data is ordered within the data structure in an order that corresponds to a sequence of genetic material in the gene.

74. The non-transitory computer readable storage medium of claim 73, wherein:
the SNP data includes a first SNP data value corresponding to a first position in the gene, and a second SNP data value corresponding to a second position in the gene, the first position in the genome having a relative position with respect to the second position in the gene, and
the first SNP data value has the relative position with respect to the second SNP data value in the data structure.

75. The non-transitory computer readable storage medium of any one of claims 73-74, wherein the SNP data for a given gene in the genome is ordered in an order that corresponds to the sequence of genetic material in the given gene in the genome.

76. The non-transitory computer readable storage medium of any one of claims 73-75, wherein the SNP data for a given pseudogene in the genome is ordered in an order that corresponds to the sequence of genetic material in the given pseudogene in the genome.

77. The non-transitory computer readable storage medium of any one of claims 73-76, wherein:
the SNP data for a given gene in the genome and the SNP data for a given pseudogene in the genome are ordered in an order that corresponds to the sequence of genetic material in the given gene and the given pseudogene in the genome.

78. The non-transitory computer readable storage medium of any one of claims 73-77, wherein the copy number data is represented in the data structure and corresponds to one or more positions in the genome, and is ordered within the data structure in an order that corresponds to an order of the one or more positions in the genome.

79. The non-transitory computer readable storage medium of claim 78, wherein:
the SNP data includes a first SNP data value corresponding to a first position in the genome,
the copy number data includes a first copy number data value corresponding to a second position in the genome,
the first position in the genome has a relative position with respect to the second position in the genome, and
the first SNP data value has the relative position with respect to the first copy number data value in the data structure.

80. The non-transitory computer readable storage medium of any one of claims 78-79, wherein the copy number data for a given gene in the genome, the copy number data for a given pseudogene in the genome, and the SNP data corresponding to the one or more positions in the genome are ordered in an order that corresponds to the sequence of genetic material in the given gene and the given pseudogene, and the one or more locations in the genome.

81. The non-transitory computer readable storage medium of any one of claims 46-80, the method further comprising determining a plurality of carrier statuses of the individual, including the respective carrier status, wherein:
the plurality of carrier statuses is represented as a data structure having an ordering characteristic, and the plurality of carrier statuses is ordered within the data structure in an order that corresponds to a sequence of genetic material in the genome.

82. The non-transitory computer readable storage medium of claim 81, wherein:
the plurality of carrier statuses includes a first carrier status associated with a first position in the genome, and a second carrier status associated with a second position in the genome, the first position in the genome having a relative position with respect to the second position in the genome, and
the first carrier status has the relative position with respect to the second carrier status in the data structure.

83. The non-transitory computer readable storage medium of any one of claims 46-82, wherein the copy number data and the SNP data are represented as a single data structure inputted to the machine learning algorithm.

84. The non-transitory computer readable storage medium of any one of claims 46-83, wherein determining the respective carrier status of the individual is further based on copy number data for a pseudogene in the genome of the individual corresponding to the gene, and SNP data for the pseudogene, wherein the machine learning algorithm is configured to further receive, as inputs, the copy number data for the pseudogene and the SNP data for the pseudogene.

85. The non-transitory computer readable storage medium of claim 84, wherein the gene and the pseudogene are selected from the group consisting of CYP21A2/CYP21A1P, GBA/psGBA, PMS2/PMS2CL and SMN1/SMN2.

86. The non-transitory computer readable storage medium of any one of claims 46-85, wherein the machine learning algorithm is trained on carrier statuses determined by human analysis (call review).

87. The non-transitory computer readable storage medium of any one of claims 46-86, wherein the machine learning algorithm is trained on carrier statuses determined by orthogonal experimental measurements.

88. The non-transitory computer readable storage medium of claim 87, wherein the orthogonal experimental measurements are long-range PCR and sequencing.

89. A non-transitory computer readable storage medium storing instructions, which when executed by one or more processors of an electronic device, cause the electronic device to perform a method for determining a number of functional copies of a gene within a genome of an individual, the method comprising:
determining the number of functional copies of the gene based on copy number data for the gene and SNP data for the gene using a machine learning algorithm, wherein the machine learning algorithm is configured to receive, as inputs, the copy number data and the SNP data, and output the number of functional copies of the gene.

90. The non-transitory computer readable storage medium of claim 89, the method further comprising any one of the methods of the non-transitory computer readable storage mediums of claims 46-88.

91. An electronic device comprising:
one or more processors; and
memory storing instructions, which when executed by the one or more processors, cause the electronic device to perform a method for determining a respective carrier status of an individual, the method comprising:
determining the respective carrier status based on copy number data for a gene in a genome of the individual and SNP data for the gene using a machine learning algorithm, wherein the machine learning algorithm is configured to receive, as inputs, the copy number data and the SNP data, and output the respective carrier status of the individual.

92. The electronic device of claim 91, wherein the gene is sequenced to determine the copy number data and the SNP data.

93. The electronic device of claim 92, wherein the gene is sequenced using direct targeted sequencing to determine the copy number data and the SNP data.

94. The electronic device of any one of claims 91-93, wherein the respective carrier status is associated with a genetic condition.

95. The electronic device of claim 94, wherein:
the genetic condition is congenital adrenal hyperplasia (CAH), and
the respective carrier status comprises one or more of V238Clstr, 30kb_del, L308X, Q319X, c332-339, P454S, c293-13, P31L, R357W, I173N and V282L carrier statuses.

96. The electronic device of any one of claims 91-95, wherein the genome comprises a gene associated with the respective carrier status and/or a pseudogene corresponding to the gene, and the copy number data is indicative of a number of copies of genetic material corresponding to the gene and/or the pseudogene that are detected during sequencing of the genome at a plurality of locations across the genome.

97. The electronic device of any one of claims 91-96, wherein the SNP data is indicative of a number of sequencing reads from the gene that have a single nucleotide polymorphism relative to a reference sequence at one or more locations across the gene.

98. The electronic device of any one of claims 91-97, wherein prior to determining the respective carrier status using the machine learning algorithm, the machine learning algorithm is trained using copy number data, SNP data and known carrier status data for one or more other genetic samples from other individuals.

99. The electronic device of claim 98, wherein the one or more other genetic samples include one or more known carrier genetic samples and one or more known non-carrier genetic samples.

100. The electronic device of any one of claims 91-99, the method further comprising determining a plurality of carrier statuses of the individual, including the respective carrier status, wherein the machine learning algorithm includes an output for each carrier status of the plurality of carrier statuses.

101. The electronic device of any one of claims 91-100, wherein:
the copy number data comprises one or more copy number values,
the SNP data comprises one or more SNP values, and
the machine learning algorithm includes an input for:
each copy number value of the one or more copy number values, and
each SNP value of the one or more SNP values.

102. The electronic device of claim 101, wherein each input of the machine learning algorithm associated with the one or more copy number values corresponds to a unique sequencing probe used to sequence the gene.

103. The electronic device of any one of claims 101-102, wherein each input of the machine learning algorithm associated with the one or more SNP values corresponds to a location in the gene associated with one or more probes used to sequence the gene.

104. The electronic device of any one of claims 91-103, wherein determining the respective carrier status comprises:
in accordance with a determination that the respective carrier status is determined with a confidence level below a confidence level threshold, flagging the determined respective carrier status for review; and
in accordance with a determination that the respective carrier status is determined with a confidence level above the confidence level threshold, forgoing flagging the determined respective carrier status for review.

105. The electronic device of claim 104, wherein determining the respective carrier status with the confidence level above the confidence level threshold includes determining that the respective carrier status determined using the machine learning algorithm is in agreement with a carrier status determination for the individual from a carrier status determination algorithm different than the machine learning algorithm.

106. The electronic device of any one of claims 91-105, the method further comprising:
prior to determining the respective carrier status of the individual, determining whether the copy number data or the SNP data are statistically anomalous; and
in accordance with a determination that the copy number data and/or the SNP data are statistically anomalous, flagging the copy number data and/or the SNP data for review.

107. The electronic device of any one of claims 91-106, the method further comprising determining a confidence level for the determination of the respective carrier status using the machine learning algorithm, wherein the machine learning algorithm is further configured to output the confidence level.

108. The electronic device of any one of claims 91-107, wherein the machine learning algorithm comprises a neural network configured to receive the copy number data and the SNP data as inputs, and output the respective carrier status of the individual.

109. The electronic device of claim 108, wherein the neural network comprises a recurrent neural network.

110. The electronic device of any one of claims 91-109, wherein the copy number data is represented as a data structure having an ordering characteristic, and the copy number data is ordered within the data structure in an order that corresponds to a sequence of genetic material in the gene.

111. The electronic device of claim 110, wherein:
the copy number data includes a first copy number data value corresponding to a first position in the gene, and a second copy number data value corresponding to a second position in the gene, the first position in the genome having a relative position with respect to the second position in the gene, and
the first copy number data value has the relative position with respect to the second copy number data value in the data structure.

112. The electronic device of any one of claims 110-111, wherein the copy number data for a given gene in the genome is ordered in an order that corresponds to the sequence of genetic material in the given gene in the genome.

113. The electronic device of any one of claims 110-112, wherein the copy number data for a given pseudogene in the genome is ordered in an order that corresponds to the sequence of genetic material in the given pseudogene in the genome.

114. The electronic device of any one of claims 110-113, wherein:
the copy number data for a given gene in the genome and the copy number data for a given pseudogene in the genome are ordered in an order that corresponds to the sequence of genetic material in the given gene and the given pseudogene in the genome.

115. The electronic device of any one of claims 110-114, wherein the SNP data is represented in the data structure and corresponds to one or more positions in the genome, and is ordered within the data structure in an order that corresponds to an order of the one or more positions in the genome.

116. The electronic device of claim 115, wherein:
the SNP data includes a first SNP data value corresponding to a first position in the genome,
the copy number data includes a first copy number data value corresponding to a second position in the genome,
the first position in the genome has a relative position with respect to the second position in the genome, and
the first SNP data value has the relative position with respect to the first copy number data value in the data structure.

117. The electronic device of any one of claims 115-116, wherein the copy number data for a given gene in the genome, the copy number data for a given pseudogene in the genome, and the SNP data corresponding to the one or more positions in the genome are ordered in an order that corresponds to the sequence of genetic material in the given gene and the given pseudogene, and the one or more locations in the genome.

118. The electronic device of any one of claims 91-109, wherein the SNP data is represented as a data structure having an ordering characteristic, and the SNP data is ordered within the data structure in an order that corresponds to a sequence of genetic material in the gene.

119. The electronic device of claim 118, wherein:
the SNP data includes a first SNP data value corresponding to a first position in the gene, and a second SNP data value corresponding to a second position in the gene, the first position in the genome having a relative position with respect to the second position in the gene, and
the first SNP data value has the relative position with respect to the second SNP data value in the data structure.

120. The electronic device of any one of claims 118-119, wherein the SNP data for a given gene in the genome is ordered in an order that corresponds to the sequence of genetic material in the given gene in the genome.

121. The electronic device of any one of claims 118-120, wherein the SNP data for a given pseudogene in the genome is ordered in an order that corresponds to the sequence of genetic material in the given pseudogene in the genome.

122. The electronic device of any one of claims 118-121, wherein:
the SNP data for a given gene in the genome and the SNP data for a given pseudogene in the genome are ordered in an order that corresponds to the sequence of genetic material in the given gene and the given pseudogene in the genome.

123. The electronic device of any one of claims 118-122, wherein the copy number data is represented in the data structure and corresponds to one or more positions in the genome, and is ordered within the data structure in an order that corresponds to an order of the one or more positions in the genome.

124. The electronic device of claim 123, wherein:
the SNP data includes a first SNP data value corresponding to a first position in the genome,
the copy number data includes a first copy number data value corresponding to a second position in the genome,
the first position in the genome has a relative position with respect to the second position in the genome, and
the first SNP data value has the relative position with respect to the first copy number data value in the data structure.

125. The electronic device of any one of claims 123-124, wherein the copy number data for a given gene in the genome, the copy number data for a given pseudogene in the genome, and the SNP data corresponding to the one or more positions in the genome are ordered in an order that corresponds to the sequence of genetic material in the given gene and the given pseudogene, and the one or more locations in the genome.

126. The electronic device of any one of claims 91-125, the method further comprising determining a plurality of carrier statuses of the individual, including the respective carrier status, wherein:
the plurality of carrier statuses is represented as a data structure having an ordering characteristic, and the plurality of carrier statuses is ordered within the data structure in an order that corresponds to a sequence of genetic material in the genome.

127. The electronic device of claim 126, wherein:
the plurality of carrier statuses includes a first carrier status associated with a first position in the genome, and a second carrier status associated with a second position in the genome, the first position in the genome having a relative position with respect to the second position in the genome, and
the first carrier status has the relative position with respect to the second carrier status in the data structure.

128. The electronic device of any one of claims 91-127, wherein the copy number data and the SNP data are represented as a single data structure inputted to the machine learning algorithm.

129. The electronic device of any one of claims 91-128, wherein determining the respective carrier status of the individual is further based on copy number data for a pseudogene in the genome of the individual corresponding to the gene, and SNP data for the pseudogene, wherein the machine learning algorithm is configured to further receive, as inputs, the copy number data for the pseudogene and the SNP data for the pseudogene.

130. The electronic device of claim 129, wherein the gene and the pseudogene are selected from the group consisting of CYP21A2/CYP21A1P, GBA/psGBA, PMS2/PMS2CL and SMN1/SMN2.

131. The electronic device of any one of claims 91-130, wherein the machine learning algorithm is trained on carrier statuses determined by human analysis (call review).

132. The electronic device of any one of claims 91-131, wherein the machine learning algorithm is trained on carrier statuses determined by orthogonal experimental measurements.

133. The electronic device of claim 132, wherein the orthogonal experimental measurements are long-range PCR and sequencing.

134. An electronic device comprising:
one or more processors; and
memory storing instructions, which when executed by the one or more processors, cause the electronic device to perform a method for determining a number of functional copies of a gene within a genome of an individual, the method comprising:
determining the number of functional copies of the gene based on copy number data for the gene and SNP data for the gene using a machine learning algorithm, wherein the machine learning algorithm is configured to receive, as inputs, the copy number data and the SNP data, and output the number of functional copies of the gene.

135. The electronic device of claim 134, the method further comprising any one of the methods of the electronic devices of claims 91-133.
